(19) United States
(12) Patent Application Publication
(10) Pub. No.: US 2022/0000336 A1
(43) Pub. Date: Jan. 6, 2022

(54) VIDEO PROCESSOR, ENDOSCOPE SYSTEM, ENDOSCOPE, AND IMAGE PROCESSING METHOD

(71) Applicant: OLYMPUS CORPORATION, Tokyo (JP)

(72) Inventors: Erika YANAGIHARA, Tokyo (JP); Shinsuke TANI, Tokyo (JP)

(73) Assignee: OLYMPUS CORPORATION, Tokyo (JP)

(21) Appl. No.: 17/380,369

(22) Filed: Jul. 20, 2021

Related U.S. Application Data
(63) Continuation of application No. PCT/JP2019/003370, filed on Jan. 31, 2019.

(51) Int. Cl. A61B 1/00; H04N 5/235

(52) U.S. Cl. CPC A61B 1/00006; H04N 5/2352; H04N 5/2351; H04N 2005/2255; A61B 1/00016; A61B 1/00032; A61B 1/00009

(57) ABSTRACT

An endoscope system includes an endoscope, a video processor, and a parameter control device. The parameter control device causes the endoscope and the video processor to execute predetermined processing by controlling a plurality of parameters used by the endoscope and the video processor. The parameter control device includes a data collection unit, an operation mode determination unit, and a parameter determination unit. The operation mode determination unit selects one or more operation modes from among an electric power consumption reducing mode, a wireless transmission amount reducing mode, a high image quality achieving mode, and a standard mode by determining the plurality of pieces of information acquired by the data collection unit. The parameter determination unit determines the plurality of parameters based on one or more operation modes selected by an operation mode selection unit.

### CROSS REFERENCE TO RELATED APPLICATION

This application is a continuation application of PCT/JP2019/003370 filed on Jan. 31, 2019, the entire contents of which are incorporated herein by, this reference.

### BACKGROUND OF THE INVENTION

### 1. Field of the Invention

The present invention relates to a video processor, an endoscope system, and an image processing method that are capable of selecting an operation mode that defines operation contents of an endoscope and the video processor.

### 2. Description of the Related Art

Recently, an endoscope device has been widely used in medical and industrial fields. In particular, an endoscope used in the medical field has been widely used for observation of an organ in a body cavity, medical treatment using a treatment instrument, a surgical operation under endoscope observation, and the like.

Recently, practical use of a battery-driven wireless endoscope on which a rechargeable battery is mounted has been started along with progress of semiconductor technologies and electric power consumption reduction due to use of an LED as an illumination light source. The wireless endoscope includes a wireless communication unit configured to perform wireless communication with a video processor, and compresses image data obtained through image pickup by an image pickup device and wirelessly transmits the compressed image data.

The wireless endoscope desirably can execute, as necessary, electric power consumption reducing control that reduces an electric power consumption of the endoscope to prevent function decrease such as battery degradation by reducing internal temperature rise and to increase an operational time by reducing a consumption amount of the battery. In addition, to prevent wireless communication blackout, the wireless endoscope desirably can execute wireless transmission amount reducing control that reduces a wireless transmission amount by increasing a compression ratio of image data in a situation in which wireless environment is degraded. It is also desirable that high image quality achieving control for obtaining an endoscope image of high image quality can be executed in an important scene.

WO 2017/029839 discloses a wireless endoscope configured to perform power saving operation that increases an image compression ratio and decreases an illumination light amount at battery replacement. Japanese Patent No. 4800695 discloses an endoscope device configured to reduce electric power consumption by controlling operation of each component of a body part of an endoscope device in accordance with internal temperature of the body part and an actual examination situation. WO 2016/052175 discloses a portable endoscope system configured to calculate a compression ratio of an endoscope image based on a result of determination of a procedure scene type. Japanese Patent No. 5649657 discloses a system configured to control power consumption of an in-vivo image pickup device configured to change a frame acquisition rate, in accordance with an amount of available energy remaining at a power source of the device.

### SUMMARY OF THE INVENTION

A video processor according to an aspect of the present invention is a video processor including a processor. The processor is configured to: acquire at least two pieces of information of information related to temperature of a grasping portion of an endoscope, information related to wireless environment of wireless communication that transmits and receives image data obtained through image pickup by the endoscope, information related to a remaining amount of a battery of the endoscope, or information for starting video recording of an endoscope image; and control a plurality of parameters. The processor selects, based on the at least two pieces of information, one or more operation modes of a plurality of operation modes that define operation contents of the endoscope and the video processor, and determines the plurality of parameters based on the one or more selected operation modes.

An endoscope system according to an aspect of the present invention is an endoscope system including an endoscope, a video processor, and a processor. The processor is configured to: acquire information related to temperature of a grasping portion of the endoscope; acquire information related to wireless environment of wireless communication that transmits and receives image data obtained through image pickup by the endoscope; acquire information related to a remaining amount of a battery of the endoscope; acquire information for starting video recording of an endoscope image; and acquire information for starting automatic diagnosis support processing. The acquisition of the information related to the wireless environment, the acquisition of the information for starting video recording of an endoscope image, and the acquisition of the information for starting automatic diagnosis support processing are executed by at least one of the endoscope or the video processor, and the acquisition of the information related to the temperature of the grasping portion and the acquisition of the information related to the remaining amount of the battery are executed by the endoscope.

An endoscope according to an aspect of the present invention is an endoscope including a processor. The processor is configured to: acquire at least two pieces of information of information related to temperature of a grasping portion of the endoscope, information related to wireless environment of wireless communication that transmits and receives image data obtained through image pickup by the endoscope, information related to a remaining amount of a battery of the endoscope, or information for starting video recording of an endoscope image; and control a plurality of parameters. The processor selects, based on the at least two pieces of information, one or more operation modes of a plurality of operation modes that define operation contents of the endoscope and a video processor, and determines the plurality of parameters based on the one or more selected operation modes,

An image processing method according to an aspect of the present invention is an image processing method of processing image data acquired by an image pickup device of an endoscope. The image processing method includes: acquiring at least two pieces of information of information related to temperature of a grasping portion of the endoscope, information related to wireless environment of wireless communication that transmits and receives the image data, information related to a remaining amount of a battery of the endoscope, or information for starting video recording of an endoscope image; selecting, based on the at least two pieces of information, one or more operation modes of a plurality of operation modes that define operation contents of the endoscope and a video processor; and determining a plurality of parameters based on the one or more selected operation modes.

### BRIEF DESCRIPTION OF THE DRAWINGS

FIG. 1 is an explanatory diagram illustrating an entire configuration of an endoscope system according to a first embodiment of the present invention;

FIG. 2 is a functional block diagram illustrating configurations of an endoscope and a parameter control device of the endoscope system according to the first embodiment of the present invention;

FIG. 3 is a functional block diagram illustrating configurations of a video processor and a display unit of the endoscope system according to the first embodiment of the present invention;

FIG. 4 is an explanatory diagram illustrating an example of a hardware configuration of the endoscope system according to the first embodiment of the present invention;

FIG. 5 is a flowchart illustrating part of operation of the endoscope system according to the first embodiment of the present invention;

FIG. 6 is a flowchart illustrating part of the operation of the endoscope system according to the first embodiment of the present invention;

FIG. 7 is a flowchart illustrating part of the operation of the endoscope system according to the first embodiment of the present invention;

FIG. 8 is a flowchart illustrating part of the operation of the endoscope system according to the first embodiment of the present invention;

FIG. 9 is a flowchart illustrating part of the operation of the endoscope system according to the first embodiment of the present invention;

FIG. 10 is a flowchart illustrating part of the operation of the endoscope system according to the first embodiment of the present invention;

FIG. 11 is an explanatory diagram schematically illustrating change of a remaining amount of a battery in the first embodiment of the present invention;

FIG. 12 is a functional block diagram illustrating configurations of an endoscope and a first part of a parameter control device in an endoscope system according to a second embodiment of the present invention; and

FIG. 13 is a functional block diagram illustrating configurations of the video processor and a second part of the parameter control device in the endoscope system according to the second embodiment of the present invention.

### DETAILED DESCRIPTION OF THE PREFERRED EMBODIMENTS

Embodiments of the present invention will be described below with reference to the accompanying drawings.

### First Embodiment

(Configuration of Endoscope System)

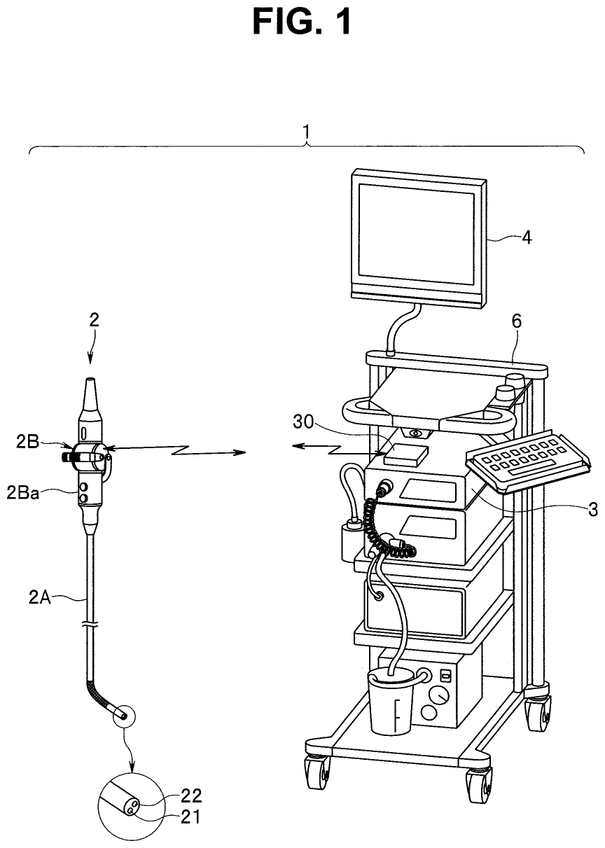

First, a schematic configuration of an endoscope system according to a first embodiment of the present invention will be described below. FIG. 1 is an explanatory diagram illustrating an entire configuration of an endoscope system 1 according to the present embodiment. The endoscope system 1 according to the present embodiment is a wireless endoscope system including a wireless endoscope 2 that is a battery-driven portable endoscope, Hereinafter, the wireless endoscope 2 is simply referred to as the endoscope 2.

The endoscope system 1 further includes a video processor 3 physically separated from the endoscope 2, and a display unit 4 connected to the video processor 3. The video processor 3 is wirelessly connected to the endoscope 2 and generates an endoscope image by performing predetermined image processing to be described later. The display unit 4 is configured of a monitor device or the like and displays the endoscope image and the like.

As illustrated in FIG. 1, the video processor 3, the display unit 4, and various medical instruments are placed on a cart 6 in an operation room, Examples of medical instruments placed on the cart 6 include devices such as an electrocautery scalpel device, a pneumoperitoneum apparatus, and a video recorder, and a gas cylinder filled with carbon dioxide.

Note that a configuration of the video processor 3 and the display unit 4 is not limited to an example illustrated in FIG. 1. For example, the endoscope system 1 may include a video processor integrated with a display unit in place of the video processor 3 and the display unit 4.

The endoscope 2 includes an elongated insertion portion 2A that is inserted into a body cavity, and an operation portion 2B including a grasping portion 2Ba that is grasped by a user. The operation portion 2B is provided at a proximal end portion of the insertion portion 2A.

The endoscope 2 further includes an image pickup unit 21 configured to generate image data through image pickup of an object, and an illumination unit 22 configured to illuminate the object. The object is a site such as an affected part in a subject. The image pickup unit 21 includes a non-illustrated image pickup device such as a CCD or a CMOS provided at a distal end portion of the insertion portion 2A.

The illumination unit 22 includes an illumination light source including a non-illustrated light-emitting element such as a light-emitting diode, and a non-illustrated lens provided at a distal end of the insertion portion 2A. Illumination light generated by the illumination light source is applied to the object through the lens. Return light of the illumination light from the object is imaged on an image pickup surface of the image pickup device of the image pickup unit 21. Note that the illumination light source may be provided in the operation portion 2B. In this case, the illumination light generated by the illumination light source is guided to the distal end of the insertion portion 2A through a non-illustrated light guide.

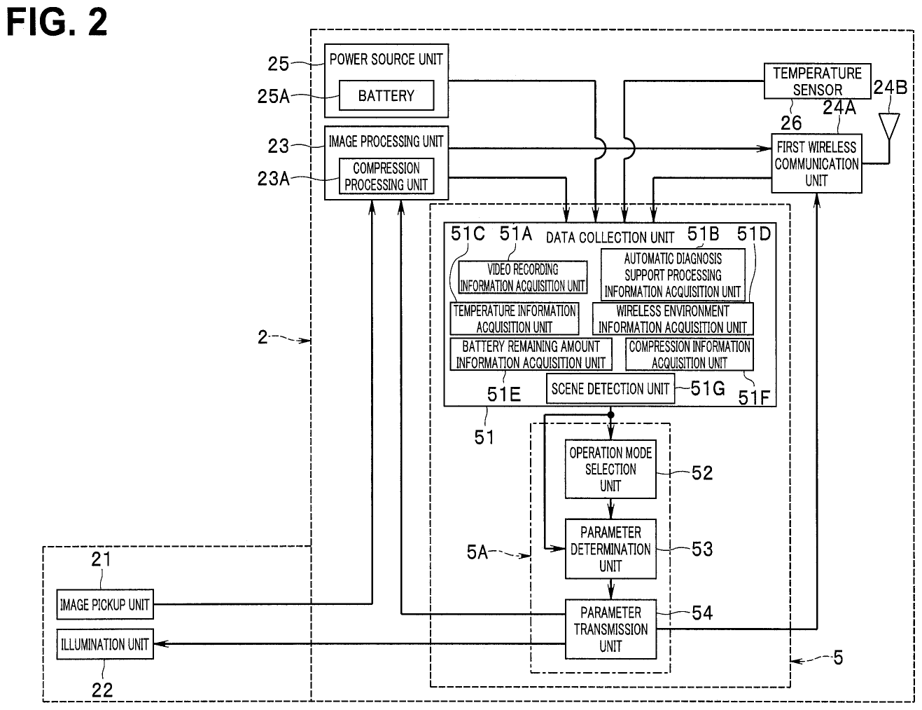

The endoscope system 1 further includes a parameter control device 5 according to the present embodiment. Note that the parameter control device 5 is illustrated in FIG. 2 to be described later. The parameter control device 5 is a device that causes the endoscope 2 and the video processor 3 to execute predetermined processing by, controlling a plurality of parameters used by, the endoscope 2 and the video processor 3.

(Configurations of Endoscope and Parameter Control Device)

Subsequently, configurations of the endoscope 2 and the parameter control device 5 will be described below in detail with reference to FIG. 2. FIG. 2 is a functional block diagram illustrating the configurations of the endoscope 2 and the parameter control device 5. In the present embodiment, the entire parameter control device 5 is provided in the endoscope 2.

As illustrated in FIG. 2, the endoscope 2 includes a first image processing unit (hereinafter simply referred to as an image processing unit) 23, a first wireless communication unit 24A, an antenna 24B, a power source unit 25, and a temperature sensor 26 in addition to the grasping portion 2Ba, the image pickup unit 21, and the illumination unit 22. The image pickup unit 21 generates image data based on an object optical image through photoelectric conversion and outputs the image data to the image processing unit 23.

The image processing unit 23 includes a compression processing unit 23A. The compression processing unit 23A performs compression processing that generates compressed data by compressing the image data generated by the image pickup unit 21. In the compression processing, a compression parameter that defines a data amount of the compressed data is used. The compression parameter has a compression ratio and a correspondence relation of the compressed data. The image processing unit 23 outputs the generated compressed data to the first wireless communication unit 24A and outputs the present compression parameter to the parameter control device 5. In addition, the image processing unit 23 outputs the image data for detecting an endoscope scene as information related to the endoscope scene to the parameter control device 5.

The first wireless communication unit 24A includes a. non-illustrated wireless transmission circuit configured to generate a wirelessly transmitted signal, and a non-illustrated wireless reception circuit configured to demodulate a wirelessly received signal. The first wireless communication unit 24A wirelessly transmits and receives a predetermined signal to and from the video processor 3 through the antenna 24B. The predetermined signal includes compressed data, and a plurality of parameters and start information to be described later.

The first wireless communication unit 24A further includes a non-illustrated environment detection circuit configured to detect a state of wireless communication environment (hereinafter simply referred to as wireless environment). The environment detection circuit detects, as the state of the wireless environment, for example, a wireless communication instrument existing in surroundings and using the same frequency band. The first wireless communication unit 24A outputs information related to the wireless environment detected by the environment detection circuit to the parameter control device 5. Note that the first wireless communication unit 24A may directly output a result of the detection by the environment detection circuit, or may calculate a forwardable data amount based on the result of the detection by the environment detection circuit and may output the calculated forwardable data amount. The forwardable data amount in wireless communication is defined in specifications of the wireless communication or changed depending on the wireless environment. The forwardable data amount is defined as, for example, a data amount that can be forwarded during a time in which image data of one frame is transmitted. The forwardable data amount decreases, for example, as the number of wireless communication instruments using the same frequency band increases.

Note that the first wireless communication unit 24A and a second wireless communication unit to be described later may be able to perform wireless communication by using a plurality of bands such as a 60-GHz band and a 5-GHz band. In this case, the 60-GHz band is used to, for example, transmit and receive compressed data. The 5-GHz band is used to, for example, transmit and receive a plurality of parameters.

The power source unit 25 includes a battery 25A and supplies electric power of the battery 25A to each component of the endoscope 2 including the image pickup unit 21, the illumination unit 22, the image processing unit 23, and the first wireless communication unit 24A. The battery 25A is mountable on, for example, the operation portion 2B (refer to FIG. 1). In addition, the power source unit 25 includes a non-illustrated battery remaining amount detection circuit configured to detect a remaining amount of the battery 25A. The power source unit 25 outputs information of the detected remaining amount of the battery 25A to the parameter control device 5.

The temperature sensor 26 is able to measure temperature of the grasping portion 2Ba (refer to FIG. 1), and outputs a measurement result of the temperature of the grasping portion 2Ba to the parameter control device 5. Note that the endoscope 2 may include, in addition to the temperature sensor 26, one or more temperature sensors configured to measure temperature of each component of the endoscope 2 except for the grasping portion 2Ba and the temperature sensor 26.

As illustrated in FIG. 2, the parameter control device 5 includes a data collection unit 51, an operation mode selection unit 52, a parameter determination unit 53, and a parameter transmission unit 54. The operation mode selection unit 52, the parameter determination unit 53, and the parameter transmission unit 54 are included in a control unit 5A as a main part of the parameter control device 5. In other words, the operation mode selection unit 52 and the parameter determination unit 53 are provided in the endoscope 2. The data collection unit 51 acquires a plurality of pieces of information related to the endoscope system 1. A configuration of the data collection unit 51 will be described later.

The operation mode selection unit 52 determines the plurality of pieces of information acquired by the data collection unit 51, thereby selecting one or more operation modes of a plurality of operation modes that each define operation contents of the endoscope 2 and the video processor 3. The parameter determination unit 53 determines a plurality of parameters based on the one or more operation modes selected by the operation mode selection unit 52. The plurality of operation modes will be described later.

The parameter transmission unit 54 transmits the plurality of parameters determined by the parameter determination unit 53 to each component of the endoscope 2 and the video processor 3. In the endoscope 2, the illumination unit 22 and the compression processing unit 23A receive the parameters transmitted from the parameter transmission unit 54. In the video processor 3, a main control unit to be described later receives the parameters transmitted from the parameter transmission unit 54.

The endoscope 2 further includes a non-illustrated main control unit. The main control unit controls each component of the endoscope 2 including the parameter control device 5, and also controls the power source unit 25 to supply power to each component of the endoscope 2 including the parameter control device 5.

(Configuration of Video Processor)

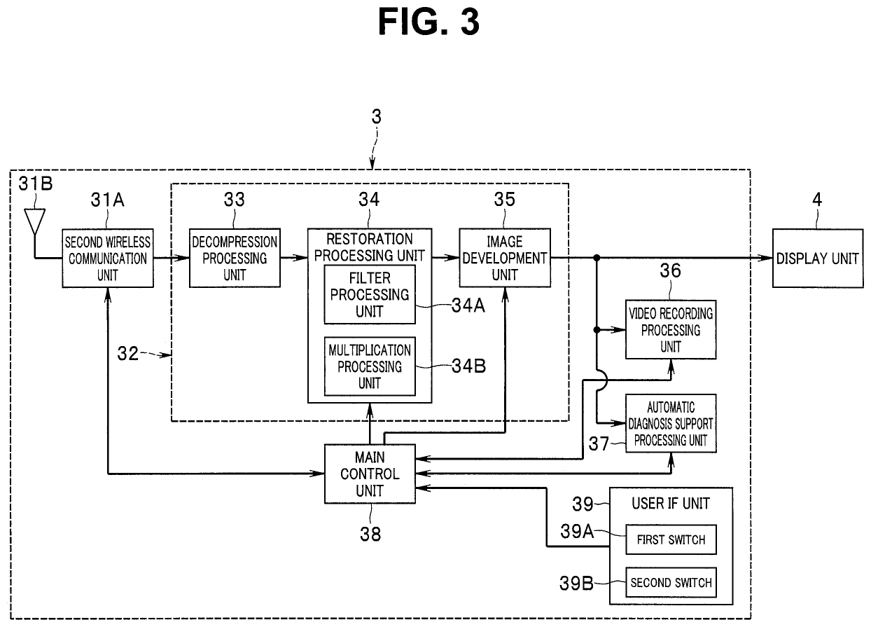

Subsequently, a configuration of the video processor 3 will be described below with reference to FIG. 3. FIG. 3 is a functional block diagram illustrating a configuration of the video processor 3 and the display unit 4. As illustrated in FIG. 3, the video processor 3 includes a second wireless communication unit 31A, an antenna 31B, a second image processing unit (hereinafter simply referred to as an image processing unit) 32, a video recording processing unit 36, an automatic diagnosis support processing unit 37, a main control unit 38. and a user interface unit (hereinafter referred to as a user IF unit) 39.

The second wireless communication unit 31A and the antenna 31B may be built in a main body of the video processor 3 or may be built in a wireless receiver 30 separated from the main body of the video processor 3. FIG. 1 illustrates the wireless receiver 30. The wireless receiver 30 is connected to the main body of the video processor 3 through a non-illustrated connector.

The second wireless communication unit 31A includes a non-illustrated wireless transmission circuit configured to generate a wirelessly transmitted signal, and a non-illustrated wireless reception circuit configured to demodulate a wirelessly received signal. The second wireless communication unit 31A wirelessly transmits and receives a predetermined signal to and from the endoscope 2 through the antenna 31B. The predetermined signal includes the compressed data transmitted by the first wireless communication unit 24A, the plurality of parameters transmitted by the parameter transmission unit 54, and the start information to be described later. The second wireless communication unit 31A outputs the compressed data to the image processing unit 32, and outputs the plurality of parameters to the main control unit 38.

The second wireless communication unit 31A may further include a non-illustrated environment detection circuit configured to detect the state of the wireless environment. Functions of the environment detection circuit of the second wireless communication unit 31A are the same as functions of the environment detection circuit of the first wireless communication unit 24A. The second wireless communication unit 31A outputs information related to the wireless environment detected by the environment detection circuit to the parameter control device 5 through wireless communication between the endoscope 2 and the video processor 3. Contents of the information related to the wireless environment and outputted from the second wireless communication unit 31A are the same as contents of the information related to the wireless environment and outputted from the first wireless communication unit 24A described above.

The image processing unit 32 generates decompressed image data corresponding to image data by decompressing the compressed data, and generates an endoscope image by performing predetermined image processing on the decompressed image data. In the present embodiment, the image processing unit 32 includes a decompression processing unit 33 configured to generate the decompressed image data, a restoration processing unit 34, and an image development unit 35.

The restoration processing unit 34 performs at least one piece of image restoration processing on the decompressed image data to improve image quality of the endoscope image. In the present embodiment, in particular, the restoration processing unit 34 is able to perform, as the at least one piece of image restoration processing, brightness correction processing that corrects brightness of the decompressed image data. Specifically, the restoration processing unit 34 includes a filter processing unit 34A and a multiplication processing unit 34B that execute the brightness correction processing.

The filter processing unit 34A performs filter processing that corrects brightness of any one pixel of the decompressed image data by using a plurality of pixel values in a predetermined region including the one pixel and a plurality of pixels surrounding the one pixel, and a first brightness parameter. The filter processing may be, for example, processing that, for each channel of RGB, multiplies values of brightness of the plurality of surrounding pixels by coefficients (weights) and adds the multiplied values to a value of brightness of the one pixel. In this case, the first brightness parameter may be the coefficients (weights) by which the values of brightness of the plurality of pixels are multiplied.

The multiplication processing unit 34B performs multiplication processing that corrects brightness of any one pixel by using a pixel value of the one pixel and a second brightness parameter. The multiplication processing may be processing that multiplies a luminance value of the one pixel by the second brightness parameter as a multiplier. In this case, the second brightness parameter may be a constant or may be a value that changes in accordance with the luminance value as in gamma correction. In the latter case, the multiplication processing is performed by using a table indicating a relation between the luminance value and the second brightness parameter.

Note that, as an effect of the filter processing is stronger, the decompressed image data after correction is brighter but a resolution of the decompressed image data after correction is lower. In addition, as an effect of the multiplication processing is stronger, the decompressed image data after correction is brighter but noise of the decompressed image data after correction is larger. Thus, in order to obtain the endoscope image of high image quality and high resolution by performing the filter processing and the multiplication processing so that the endoscope image becomes brighter, it is needed to set the first brightness parameter to avoid excess decrease of the resolution of the decompressed image data after correction, and to set the second brightness parameter to avoid excess increase of the noise of the decompressed image data after correction.

The image development unit 35 performs image development processing that generates the endoscope image by convening the decompressed image data into a format displayable on the display unit 4. The image processing unit 32 outputs the generated endoscope image to the video recording processing unit 36, the automatic diagnosis support processing unit 37, and the display unit 4.

The user IF unit 39 is an interface configured to receive a user operation. Specifically, the user IF unit 39 includes, for example, a front panel and various switches of a control system, and outputs an operation signal based on the user operation to the main control unit 38. Examples of the user operation include activation of the endoscope system 1, power-off of the endoscope system 1, start and stop of video recording of the endoscope image, start and stop of automatic diagnosis support processing, specification of an observation mode of the endoscope 2, setting related to image display, and setting of an operation mode of the endoscope 2.

In the present embodiment, in particular, the user IF unit 39 includes a first switch 39A through which start and stop of the video recording of the endoscope image are instructed, and a second switch 39B through which start and stop of the automatic diagnosis support processing are instructed. An operation signal that instructs start or stop of the video recording of the endoscope image is generated as the user operates the first switch 39A. An operation signal that instructs start or stop of the automatic diagnosis support processing is generated as the user operates the second switch 39B.

The main control unit 38 controls each component of the video processor 3 and also controls a non-illustrated power source unit provided in the video processor 3 to supply power to each component of the video processor 3. The main control unit 38 receives a parameter transmitted from the parameter transmission unit 54 and outputs the received parameter to the restoration processing unit 34. The main control unit 38 outputs information based on an operation signal inputted through the user IF unit 39 to each component of the video processor 3, and also outputs the information to the non-illustrated main control unit of the endoscope 2 through wireless communication between the endoscope 2 and the video processor 3. Accordingly, the main control unit 38 can provide various instructions based on an operation signal to each component of the endoscope 2, the video processor 3, and the parameter control device 5.

In the present embodiment, in particular, the main control unit 38 generates, based on an operation signal that instructs start or stop of the video recording of the endoscope image, information for starting the video recording of the endoscope image and information for stopping the video recording of the endoscope image, and outputs these pieces of information to the video recording processing unit 36 and the parameter control device 5. In addition, the main control unit 38 generates, based on an operation signal that instructs start or stop of the automatic diagnosis support processing, information for starting the automatic diagnosis support processing and information for stopping the automatic diagnosis support processing, and outputs these pieces of information to the automatic diagnosis support processing unit 37 and the parameter control device 5. The information for starting the video recording of the endoscope image and the information for starting the automatic diagnosis support processing are referred to as start information, in particular.

The video recording processing unit 36 performs video recording processing that video-records the endoscope image generated by the image development unit 35. In the present embodiment, the video recording processing unit 36 starts the video recording processing when the information for starting the video recording of the endoscope image is inputted, and stops the video recording processing when the information for stopping the video recording of the endoscope image is inputted. Note that, after outputting the information for starting the video recording of the endoscope image to the video recording processing unit 36, the main control unit 38 controls the image development unit 35 to output the endoscope image to the video recording processing unit 36. The video recording processing unit 36 includes a non-illustrated storage unit configured to store the endoscope image video-recorded by the video recording processing. The video recording processing unit 36 may be able to output the endoscope image stored in the storage unit to the display unit 4 and a non-illustrated storage device configured of anon-transitory memory.

The endoscope image video-recorded by the video recording processing is used, for example, for production of a diagnosis report or for detailed diagnosis to be performed later. To improve accuracy of the detailed diagnosis, the endoscope image video-recorded by the video recording processing needs to be an image of high image quality.

The automatic diagnosis support processing unit 37 performs the automatic diagnosis support processing using the endoscope image. In the present embodiment, the automatic diagnosis support processing unit 37 starts the automatic diagnosis support processing when the information for starting the automatic diagnosis support processing is inputted, and stops the automatic diagnosis support processing when the information for stopping the automatic diagnosis support processing is inputted. Note that, after outputting the information for starting the automatic diagnosis support processing to the video recording processing unit 36, the main control unit 38 controls the image development unit 35 to output the endoscope image to the automatic diagnosis support processing unit 37. The automatic diagnosis support processing unit 37 may be able to output a result of the automatic diagnosis support processing to the display unit 4.

The automatic diagnosis support processing is, for example, processing that automatically detects existence of anomaly by analyzing the endoscope image generated by the image development unit 35 through image processing or the like. The analysis of the endoscope image is performed by, for example, image processing using artificial intelligence. To improve accuracy of automatic diagnosis, the endoscope image used in the automatic diagnosis support processing needs to be an image of high image quality.

(Hardware Configuration)

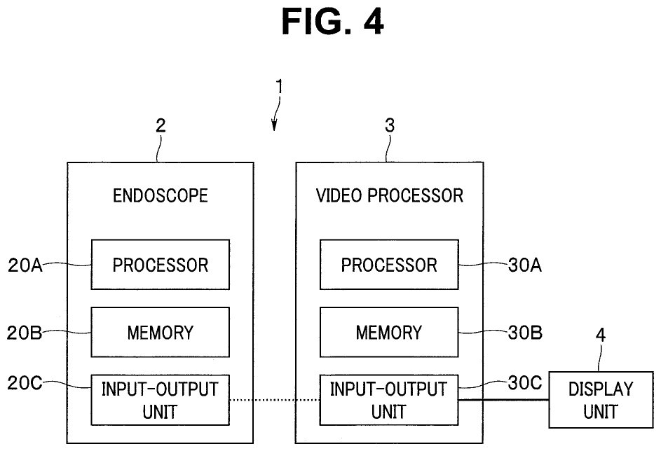

Subsequently, a hardware configuration of the endoscope system 1 will be described below with reference to FIG. 4. FIG. 4 is an explanatory diagram illustrating an example of the hardware configuration of the endoscope system 1. In the example illustrated in FIG. 4, the endoscope 2 includes a processor 20A, a memory 20B, and an input-output unit 20C. The video processor 3 includes a processor 30A, a memory 30B, and an input-output unit 30C.

The processor 20A is used to execute functions of the image processing unit 23, the first wireless communication unit 24A, the power source unit 25, the non-illustrated main control unit, and the like as components of the endoscope 2, and functions of the data collection unit 51, the operation mode selection unit 52, the parameter determination unit 53, and the parameter transmission unit 54 as components of the parameter control device 5. The processor 30A is used to execute functions of the second wireless communication unit 31A, the image processing unit 32, the main control unit 38, and the like as components of the video processor 3. The processors 20A and 30A are each configured of, for example, a field programmable gate array (FPGA). At least some of a plurality of components of the endoscope 2, the video processor 3, and the parameter control device 5 may be configured as circuit blocks in the FPGA.

The memories 20B and 30B are each configured of a rewritable storage element such as RAM. The input-output unit 20C is used to perform signal transmission and reception between the endoscope 2 and outside. The input-output unit 30C is used to perform signal transmission and reception between the video processor 3 and outside. In the present embodiment, in particular, wireless signal transmission and reception between the endoscope 2 and the video processor 3 are performed by using the input-output units 20C and 30C.

Note that the processors 20A and 30A may he each configured of a central processing unit (hereinafter referred to as a CPU). In this case, the functions of components of the endoscope 2 and the parameter control device 5 may he achieved as the CPU reads a program from the memory 20B or a non-illustrated storage device and executes the program. Similarly, the functions of components of the video processor 3 may be achieved as the CPU reads a program from the memory 30B or a non-illustrated storage device and executes the program.

The hardware configuration of the endoscope system 1 is not limited to the example illustrated in FIG. 4. For example, a plurality of components of the endoscope 2, the video processor 3, and the parameter control device 5 may be each configured as a separate electronic circuit.

(Operation of Parameter Control Device)

Subsequently, operation of the parameter control device 5 will be described below.

(Configuration and Operation of Data Collection Unit)

First, a configuration and operation of the data collection unit 51 will be described below with reference to FIG. 2. The data collection unit 51 acquires at least two pieces of information of information related to the temperature of the grasping portion 2Ba, information related to the wireless environment between the first wireless communication unit 24A and the second wireless communication unit 31A, information related to the remaining amount of the battery 25A, the information for starting the video recording of the endoscope image, and the information for starting the automatic diagnosis support processing. The following description will be on an example in which the data collection unit 51 acquires all of the above-described information.

The data collection unit 51 further acquires the infomiation for stopping the video recording of the endoscope image and the information for stopping the automatic diagnosis support processing.

In the present embodiment, the data collection unit 51 includes a video recording information acquisition unit 51A, an automatic diagnosis support processing information acquisition unit 51B, a temperature information acquisition unit 51C, a wireless environment information acquisition unit 51D, and a battery remaining amount information acquisition unit 51E. In other words, the video recording information acquisition unit 51A, the automatic diagnosis support processing information acquisition unit 51B, the temperature information acquisition unit 51C, the wireless environment information acquisition unit 51D, and the battery remaining amount information acquisition unit 51E are provided in the endoscope 2.

The video recording information acquisition unit 51A acquires the information for starting the video recording of the endoscope image and the information for stopping the video recording of the endoscope image. In the present embodiment, the video recording information acquisition unit 51A receives the information for starting the video recording of the endoscope image and the information for stopping the video recording of the endoscope image, which are outputted from the main control unit 38 (refer to FIG. 3) of the video processor 3.

The automatic diagnosis support processing information acquisition unit 51B acquires the information for starting the automatic diagnosis support processing and the information for stopping the automatic diagnosis support processing. In the present embodiment, the automatic diagnosis support processing information acquisition unit 51B receives the information for starting the automatic diagnosis support processing and the information for stopping the automatic diagnosis support processing, which are outputted from the main control unit 38 (refer to FIG. 3) of the video processor 3.

The temperature information acquisition unit 51C acquires the information related to the temperature of the grasping portion 2Ba. In the present embodiment, the temperature information acquisition unit 51C receives the measurement result of the temperature of the grasping portion 2Ba, which is outputted from the temperature sensor 26.

The wireless environment information acquisition unit 51D acquires the information related to the wireless environment. In the present embodiment, the wireless environment information acquisition unit 51D receives, the information related to the wireless environment, which is outputted from the first wireless communication unit 24A. The wireless environment information acquisition unit 51D acquires, as the information related to the wireless environment, the result of the detection by the environment detection circuit of the first wireless communication unit 24A or the forwardable data amount calculated based on the result of the detection by the environment detection circuit. When the wireless environment information acquisition unit 51D acquires the result of the detection by the environment detection circuit, the wireless environment information acquisition unit 51D may calculate the forwardable data amount based on the result of the detection by the environment detection circuit.

Note that when the second wireless communication unit 31A includes an environment detection circuit as described above, the wireless environment information acquisition unit 51D may receive the information related to the wireless environment, which is outputted from the second wireless communication unit 31A. In this case, the information related to the wireless environment, which is acquired by the wireless environment information acquisition unit 51D may be information outputted from the first wireless communication unit 24A or may be information outputted from the second wireless communication unit 31A.

The battery remaining amount information acquisition unit 51E acquires the information related to the remaining amount of the battery 25A. In the present embodiment, the battery remaining amount information acquisition unit 51E receives the information related to the remaining amount of the battery 25A, which is outputted from the power source unit 25.

The data collection unit 51 further includes a compression information acquisition unit 51F and a scene detection unit 51G. The compression information acquisition unit 51F acquires information related to the compression processing. In the present embodiment, the compression information acquisition unit 51F receives the compression parameter outputted from the image processing unit 23.

The scene detection unit 51G acquires information related to an endoscope scene. In the present embodiment, image data for detecting an endoscope scene is outputted from the image processing unit 23 and inputted to the scene detection unit 51G. The scene detection unit 51G detects an endoscope scene by analyzing the image data. Examples of the endoscope scene include a detailed-check scene corresponding to a case of detailed-check observation of a blood vessel or the like, a screening scene corresponding to, for example, a case of search for an anomalous part, while moving the insertion portion 2A, and an external scene corresponding to a case of external positioning of the insertion portion 2A.

(Operation of Operation Mode Selection Unit)

Subsequently, operation of the control unit 5A of the parameter control device 5, in other words, operation of the operation mode selection unit 52, the parameter determination unit 53, and the parameter transmission unit 54 will be described below with reference to FIGS. 2 and 3. First, the operation of the operation mode selection unit 52 will be described below. The operation mode selection unit 52 determines at least two pieces of information acquired by the data collection unit 51, thereby selecting one or more operation modes. In the present embodiment, in particular, the operation mode selection unit 52 determines all of the information acquired by the data collection unit 51.

In the present embodiment, the plurality of operation modes that define the operation contents of the endoscope 2 and the video processor 3 include an electric power consumption reducing mode, a wireless transmission amount reducing mode, a high image quality achieving mode, and a standard mode.

The electric power consumption reducing mode is an operation mode in which electric power consumption reducing control is performed to control the endoscope 2 and the video processor 3 to reduce electric power supplied from the battery 25A. The operation mode selection unit 52 determines whether the temperature of the grasping portion 2Ba is equal to or higher than a predetermined temperature threshold value and whether the remaining amount of the battery 25A is smaller than a predetermined battery threshold value. The operation mode selection unit 52 selects the electric power consumption reducing mode when at least one of a condition that the temperature of the grasping portion 2Ba is equal to or higher than the predetermined first temperature threshold value or a condition that the remaining amount of the battery 25A is smaller than the predetermined first battery threshold value is satisfied.

The wireless transmission amount reducing mode is an operation mode in which wireless transmission amount reducing control is performed to control the endoscope 2 and the video processor 3 to reduce an amount of data transmitted from the first wireless communication unit 24A to the second wireless communication unit 31A. The operation mode selection unit 52 determines whether the forwardable data amount is smaller than a predetermined threshold value, thereby determining whether the wireless environment is degraded. Note that when the wireless environment information acquisition unit 51D acquires or calculates the forwardable data amount, the operation mode selection unit 52 uses the forwardable data amount acquired or calculated by the wireless environment information acquisition unit 51D. When the wireless environment information acquisition unit 51D acquires the result of the detection by the environment detection circuit but does not calculate the forwardable data amount, the operation mode selection unit 52 calculates the forward.able data amount by using the result of the detection by the environment detection circuit, which is acquired by the wireless environment information acquisition unit 51D. The operation mode selection unit 52 selects the wireless transmission amount reducing mode when the forwardable data amount is smaller than the predetermined threshold value.

The high image quality achieving mode is an operation mode in which high image quality achieving control is performed to control the endoscope 2 and the video processor 3 to achieve high image quality of the endoscope image. The operation mode selection unit 52 determines whether the information for starting the video recording of the endoscope image is acquired by the video recording information acquisition unit 51A and whether the information for starting the automatic diagnosis support processing is acquired by the automatic diagnosis support processing information acquisition unit 51B. The operation mode selection unit 52 selects the high image quality achieving mode when at least one of these two pieces of information is acquired.

The standard mode is an operation mode in which neither the electric power consumption reducing control, the wireless transmission amount reducing control, nor the high image quality achieving control is performed but standard control is performed to control the endoscope 2 and the video processor 3. The operation mode selection unit 52 selects the standard mode when none of selection conditions of the electric power consumption reducing mode, the wireless transmission amount reducing mode, and the high image quality achieving mode are satisfied. The operation mode selection unit 52 may determine the information related to an endoscope scene, which is acquired by the scene detection unit 51G, thereby determining contents of the standard control.

Note that a case in which the selection condition of the high image quality achieving mode is not satisfied includes a case in which the data collection unit 51 acquires neither the information for starting the video recording of the endoscope image nor the information for starting the automatic diagnosis support processing, as well as a case in which the video recording information acquisition unit 51A acquires the information for stopping the video recording of the endoscope image during execution of the video recording processing, and a case m which the automatic diagnosis support processing information acquisition unit 51B acquires the information for stopping the automatic diagnosis support processing during execution of the automatic diagnosis support processing. The operation mode selection unit 52 may receive information of whether the video recording processing is in execution and information of whether the automatic diagnosis support processing is in execution. These pieces of information may be outputted from, for example, the main control unit 38 of the video processor 3. Alternatively, the operation mode selection unit 52 may determine whether the video recording processing is in execution based on the information for starting or stopping the video recording of the endoscope image, which is acquired by the data collection unit 51. Similarly, the operation mode selection unit 52 may determine whether the automatic diagnosis support processing is in execution based on the information for starting or stopping the automatic diagnosis support processing, which is acquired by the data collection unit 51.

(Operation of Parameter Determination Unit)

Subsequently, the operation of the parameter determination unit 53 will be described below. First, contents of the electric power consumption reducing control, the wireless transmission amount reducing control, and the high image quality achieving control will be described below through comparison with the standard control. In the following description, the standard control when the endoscope scene is the detailed-check scene is a reference. The electric power consumption reducing control and the high image quality achieving control each include illumination light amount change processing that changes an illumination light amount of the illumination unit 22, compression amount change processing that changes the data amount of the compressed data, and the brightness correction processing. The wireless transmission amount reducing control includes the compression amount change processing and the brightness correction processing.

The illumination light amount change processing is processing in which an illumination parameter that defines the illumination light amount of the illumination unit 22 is used. The illumination parameter in the electric power consumption reducing control is defined so that the illumination light amount is smaller than in the standard control. The illumination parameter in the high image quality achieving control is defined so that the illumination light amount is larger than in the standard control.

The compression amount change processing is processing in which the compression parameter that defines the data amount of the compressed data is used. The compression parameter in the electric power consumption reducing control and the compression parameter in the wireless transmission amount reducing control are defined so that the data amount of the compressed data is smaller than in the standard control. The compression parameter in the high image quality achieving control is defined so that the data amount of the compressed data is larger than in the standard control.

The brightness correction processing is processing in which a brightness parameter that defines a relation between the brightness of the decompressed image data before correction and the brightness of the decompressed image data after correction is used. The brightness parameter in the electric power consumption reducing control is defined so that an effect of the brightness correction processing that brightens the endoscope image is stronger than in the standard control. The brightness parameter in the wireless transmission amount reducing control is defined so that brightness of the endoscope image is corrected with decrease of a resolution of the endoscope image being reduced as compared to the standard control. The brightness parameter in the high image quality achieving control is defined so that the effect of the brightness correction processing is weaker than in the standard control.

In the present embodiment, the brightness parameter is the first brightness parameter used in the filter processing and the second brightness parameter used in the multiplication processing. The first brightness parameter in the electric power consumption reducing control is defined so that an effect of the filter processing is stronger than in the standard control. The second brightness parameter in the electric power consumption reducing control is defined so that the effect of the multiplication processing is stronger than in the standard control.

The first brightness parameter in the wireless transmission amount reducing control is defined so that the effect of the filter processing is weaker than in the standard control. The second brightness parameter in the wireless transmission amount reducing control is defined so that an effect of the multiplication processing is stronger than in the standard control.

The first brightness parameter in the high image quality achieving control is defined so that the effect of the filter processing is weaker than in the standard control. The second brightness parameter in the high image quality achieving control is defined so that the effect of the multiplication processing is weaker than in the standard control.

Hereinafter, the illumination parameter, the compression parameter, and the first and second brightness parameters in the electric power consumption reducing control are also referred to as Bp, Cp, Fp, and Mp, respectively. The compression parameter and the first and second brightness parameters in the wireless transmission amount reducing control are also referred to as Cw, Fw, and Mw, respectively. The illumination parameter, the compression parameter, and the first and second brightness parameters in the high image quality achieving control are also referred to as Bh, Ch, Fh, and Mh, respectively. The illumination parameter, the compression parameter, and the first and second brightness parameters in the standard control are also referred to as Bs, Cs, Fs, and Ms, respectively. These parameters are defined in advance. These parameters may be fixed values or may be values that change in accordance with contents of image data. These parameters may be stored in a non-illustrated storage device provided in the endoscope 2 or the parameter control device 5.

Subsequently, the operation of the parameter determination unit 53 will he specifically described below. First, a case in which the operation mode selection unit 52 selects any one of the electric power consumption reducing mode, the wireless transmission amount reducing mode, the high image quality achieving mode, and the standard mode will he described below. The parameter determination unit 53 determines Bp, Cp, Fp, and Mp as the plurality of parameters when the operation mode selection unit 52 selects the electric power consumption reducing mode. The parameter determination unit 53 determines Cw, Fw, and Mw as the plurality of parameters when the operation mode selection unit 52 selects the wireless transmission amount reducing mode. The parameter determination unit 53 determines Bh, Ch, Fh, and Mh as the plurality of parameters when the operation mode selection unit 52 selects the high image quality achieving mode. The parameter determination unit 53 determines Bs, Cs, Fs, and Ms as the plurality of parameters when the operation mode selection unit 52 selects the standard mode.

Note that the wireless transmission amount reducing control does not include the illumination light amount change processing. Thus, the illumination parameter is not changed when the operation mode selection unit 52 selects the wireless transmission amount reducing mode. The parameter determination unit 53 may determine the illumination parameter in the wireless transmission amount reducing control so that the illumination parameter is not changed in effect. The illumination parameter in the wireless transmission amount reducing control may be the same as Bs.

Subsequently, a case in which the operation mode selection unit 52 selects the electric power consumption reducing mode and the high image quality achieving mode will be described below. In this case, the operation of the parameter determination unit 53 differs in accordance with the temperature of the grasping portion 2Ba or the remaining amount of the battery 25A. Specifically, the parameter determination unit 53 determines the plurality of parameters in the high image quality achieving control, namely, Bh, Ch, Fh, and Mh as the plurality of parameters when the temperature of the grasping portion 2Ba is equal to or higher than a first temperature threshold value and is lower than a second temperature threshold value higher than the first temperature threshold value. The parameter determination unit 53 determines the plurality of parameters in the electric power consumption reducing control, namely, Bp, Cp, Fp, and Nip as the plurality of parameters when the temperature of the grasping portion 2Ba is equal to or higher than the second temperature threshold value.

Similarly, the parameter determination unit 53 determines the plurality of parameters in the high image quality achieving control, namely, Bh, Ch, Fh, and Mh as the plurality of parameters when the remaining amount of the battery 25.A is smaller than a first battery threshold value and is equal to or larger than a second battery threshold value smaller than the first battery threshold value. The parameter determination unit 53 determines the plurality of parameters in the electric power consumption reducing control, namely, Bp, Cp, Fp, and Mp as the plurality of parameters when the remaining amount of the battery 25A is smaller than the second battery threshold value.

Subsequently, a case in which the operation mode selection unit 52 selects the wireless transmission amount reducing mode and the high image quality achieving mode will be described below. In this case, the parameter determination unit 53 determines the illumination parameter and the first and second brightness parameters in the high image quality achieving control, namely, Bh, Fh, and Mh, and the compression parameter in the wireless transmission amount reducing control, namely, Cw as the plurality of parameters.

Subsequently, a case in which the operation mode selection unit 52 selects the electric power consumption reducing mode, the wireless transmission amount reducing mode, and the high image quality achieving mode will be described below. In this case, the operation of the parameter determination unit 53 differs in accordance with the temperature of the grasping portion 2Ba or the remaining amount of the battery 25A. Specifically, the parameter determination unit 53 determines the illumination parameter and the first and second brightness parameters in the high image quality achieving control, namely, Bh, Fh, and Mh, and the compression parameter in the wireless transmission amount reducing control, namely, Cw as the plurality of parameters when the temperature of the grasping portion 2Ba is equal to or higher than the first temperature threshold value and lower than the second temperature threshold value. The parameter determination unit 53 determines the illumination parameter and the first and second brightness parameters in the electric power consumption reducing control, namely, Bh, Fp, and Mp, and the compression parameter in the wireless transmission amount reducing control, namely, Cw as the plurality of parameters when the temperature of the grasping portion 2Ba is equal to or higher than the second temperature threshold value.

Similarly, the parameter determination unit 53 determines the illumination parameter and the first and second brightness parameters in the high image quality achieving control, namely, Bh, Fh, and Mh, and the compression parameter in the wireless transmission amount reducing control, namely, Cw as the plurality of parameters when the remaining amount of the battery 25A is smaller than the first battery threshold value and equal to or larger than the second battery threshold value. The parameter determination unit 53 determines the illumination parameter and the first and second brightness parameters in the electric power consumption reducing control, namely, Bp, Fp, and Mp, and the compression parameter in the wireless transmission amount reducing control, namely, Cw as the plurality of parameters when the remaining amount of the battery 25A is smaller than the second battery threshold value.

Note that the compression parameter can change in accordance with contents of image data. In the present embodiment, the parameter determination unit 53 receives the compression parameter acquired by the compression information acquisition unit 51F. The parameter determination unit 53 may determine the compression parameter used in the next compression processing based on a result of the operation mode selection by the operation mode selection unit 52 and the compression parameter used in the compression processing right before.

(Operation of Parameter Transmission Unit)

Subsequently, the operation of the parameter transmission unit 54 will be described below. The parameter transmission unit 54 transmits the illumination parameter to the illumination unit 22, transmits the compression parameter to the compression processing unit 23A, and transmits the first and second brightness parameters to the main control unit 38 of the video processor 3. The illumination unit 22 changes the illumination light amount of the illumination unit 22 based on the received illumination parameter. The compression processing unit 23A performs the compression processing by using the received compression parameter.

The main control unit 38 outputs the received first brightness parameter to the filter processing unit 34A of the restoration processing unit 34, and outputs the received second brightness parameter to the multiplication processing unit 3413 of the restoration processing unit 34. The filter processing unit 34A performs the filter processing by using the first brightness parameter. The multiplication processing unit 34B performs the multiplication processing by using the second brightness parameter.

(A Series of Operations Related to Parameter Control Device)

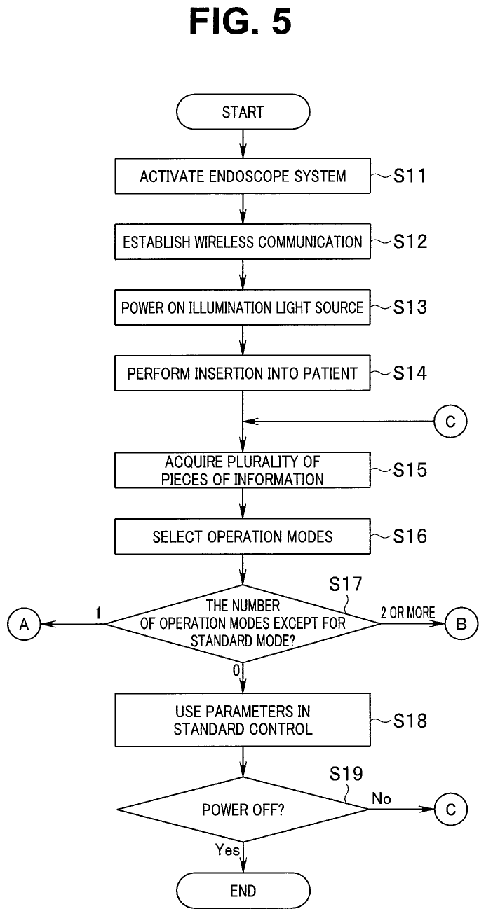

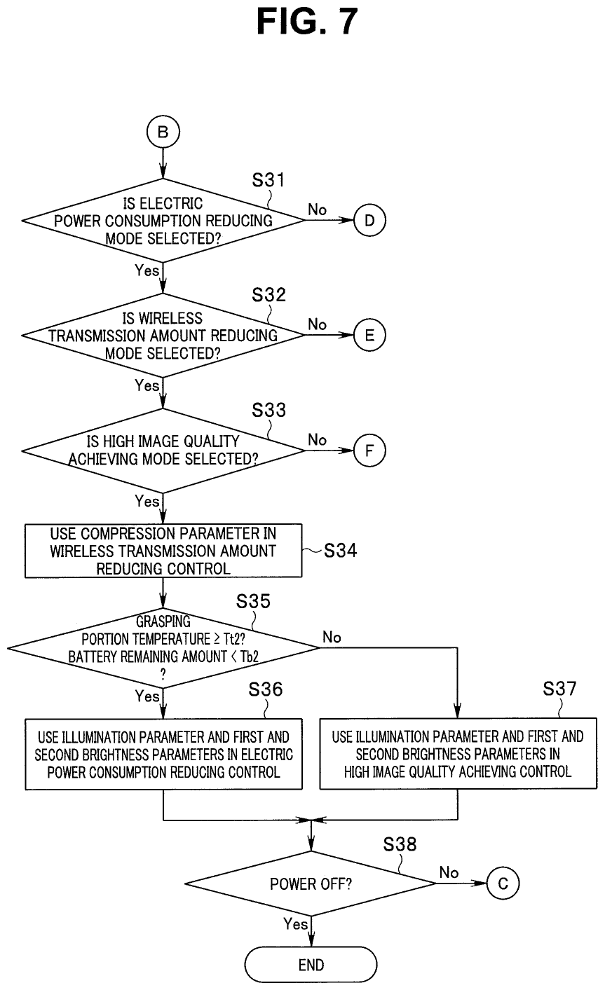

Subsequently, a specific example of a series of operations related to the parameter control device 5 in operation of the endoscope system 1 will he described below with reference to FIGS. 2, 3, and 5 to 10. FIGS. 5 to 10 are flowcharts illustrating part of the operation of the endoscope system 1. In FIGS. 7 and 9, symbol Tt2 represents the second temperature threshold value, and symbol Tb2 represents the second battery threshold value.

As illustrated in FIG. 5, first in the series of operations, an operation signal that activates the endoscope system 1 is inputted to the main control unit 38 through the user IF unit 39 as, for example, the user operates a switch or the like for activating the endoscope system 1. The main control unit 38 activates the endoscope system 1 based on the inputted operation signal (step S11). Subsequently, wireless communication connection between the endoscope 2 and the video processor 3 is established as the main control unit of the endoscope 2 controls the first wireless communication unit 24A and the main control unit 38 of the video processor 3 controls the second wireless communication unit 31A (step S12).

Subsequently, the illumination light source is powered on as the main control unit of the endoscope 2 controls the illumination unit 22 (step S13), and the endoscope 2 and the video processor 3 starts execution of the standard control. Subsequently, the user starts an insertion operation that inserts the insertion portion 2A of the endoscope 2 into a body of a patient (step S14).

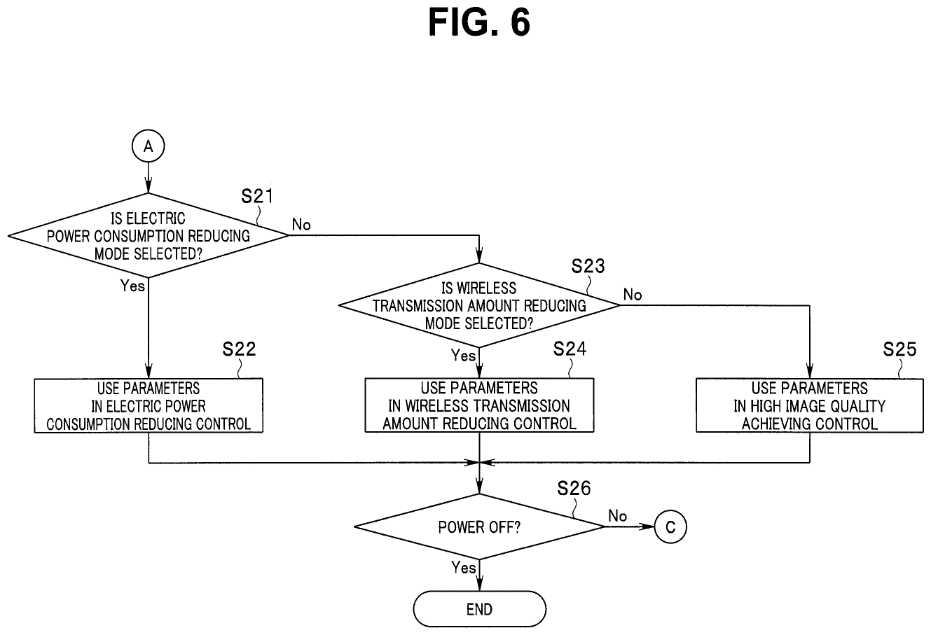

Subsequently, the data collection unit 51 acquires a plurality of pieces of information related to the endoscope system 1 (step S15). Subsequently, the operation mode selection unit 52 selects one or more operation modes (step S16). In an example illustrated in FIGS. 5 to 10, the series of operations are changed in accordance with the number of operation modes except for the standard mode, which are selected at step S16. Specifically, step S18 is executed when the number of operation modes except for the standard mode is zero, step S21 in FIG. 6 is executed when the number of operation modes except for the standard mode is one, or step S31 in FIG. 7 is executed when the number of operation modes except for the standard mode is two or more (step S17).

When the number of operation modes except for the standard mode is zero, in other words, when the operation mode selection unit 52 selects the standard mode, components of the endoscope 2 and the video processor 3 use the plurality of parameters in the standard control, namely, Bs, Cs, Fs, and Ms (step S18).

Step S18 and a step similar to step S18 are achieved as the parameter determination unit 53 determines the plurality of parameters and the parameter transmission unit 54 transmits the plurality of parameters to components of the endoscope 2 and the video processor 3 as described above. Note that the above-described operation of the parameter determination unit 53 and the parameter transmission unit 54 may be omitted when the operation mode selection unit 52 selects the standard mode in a situation in which the standard control is executed.

After step S18 is executed, for example, the main control unit 38 determines whether to power off the endoscope system 1 (step S19). Specifically, the main control unit 38 determines whether an operation signal that powers off the endoscope system 1 is inputted. The operation signal is inputted to the main control unit 38 through the user IF unit 39 as, for example, the user operates a switch or the like for powering off the endoscope system 1. When the operation signal is not inputted to the main control unit 38, the main control unit 38 determines that the endoscope system 1 is not to be powered off (No), and step S15 is executed again. When the operation signal is inputted to the main control unit 38., the main control unit 38 determines that the endoscope system 1 is to be powered off (Yes), and the series of operations are ended.

A series of steps illustrated in FIG. 6 correspond to operation of the endoscope system 1 when the number of operation modes except for the standard mode, which are selected by the operation mode selection unit 52 at step S16 is one. When the electric power consumption reducing mode is selected at step S16 (Yes at step S21), components of the endoscope 2 and the video processor 3 use the plurality of parameters in the electric power consumption reducing control, namely, Bp, Cp, Fp, and Mp (step S22).

When the electric power consumption reducing mode is not selected at step S16 (No at step S21) but the wireless transmission amount reducing mode is selected (Yes at step S23), components of the endoscope 2 and the video processor 3 use the plurality of parameters in the wireless transmission amount reducing control, namely, Bw, Cw, and Mw (step S24).

When the electric power consumption reducing mode is not selected at step S16 (No at step S21) and the wireless transmission amount reducing mode is not selected (No at step S23), in other words, when the high image quality achieving mode is selected at step S16, components of the endoscope 2 and the video processor 3 use the plurality of parameters in the high image quality achieving control, namely, Bh, Ch, Fh, and Mh (step S25).

After step S22, S24, or S25 is executed, for example, the main control unit 38 determines whether to power off the endoscope system 1 (step S26). Contents of step S26 are the same as contents of step S19 in FIG. 5. When the main control unit 38 determines that the endoscope system 1 is not to be powered off (No), step S15 in FIG. 5 is executed again. When the main control unit 38 determines that the endoscope system 1 is to he powered off (Yes), the series of operations are ended.

A series of steps illustrated in each of FIGS. 7 to 10 correspond to operation of the endoscope system 1 when the number of operation modes except for the standard mode, which are selected by the operation mode selection unit 52 at step S16 is two or more. When the electric power consumption reducing mode is selected (Yes at step S31), the wireless transmission amount reducing mode is selected (Yes at step S32), and the high image quality achieving mode is selected (Yes at step S33) at step S16, the compression processing unit 23A uses the compression parameter in the wireless transmission amount reducing control, namely, Cw (step S22).

When at least one of a requirement that the temperature of the grasping portion 2Ba, which is acquired at step S15 in FIG. 5 is equal to or higher than the second temperature threshold value Tt2 or a requirement that the remaining amount of the battery 25A, which is acquired at step S15 in FIG. 5 is smaller than the second battery threshold value Tb2 is satisfied (Yes at step S35), the illumination unit 22 uses the illumination parameter in the electric power consumption reducing control, namely, Bp, the filter processing unit 34A uses the first brightness parameter in the electric power consumption reducing control, namely, Fp, and the multiplication processing unit 34B uses the second brightness parameter in the electric power consumption reducing control, namely, Mp (step S36).

When the requirement that the temperature of the grasping portion 2Ba is equal to or higher than the second temperature threshold value Tt2 and the requirement that the remaining amount of the battery 25A is smaller than the second battery threshold value Tb2 are both not satisfied (No at step S35), the illumination unit 22 uses the illumination parameter in the high image quality achieving control, namely, Bh, the filter processing unit 34A uses the first brightness parameter in the high image quality achieving control, namely, Fh, and the multiplication processing unit 34B uses the second brightness parameter in the high image quality achieving control, namely, Mh (step S37),

After step S36 or S37 is executed, for example, the main control unit 38 determines whether to power off the endoscope system 1 (step S38). Contents of step S38 are the same as the contents of step S19 in FIG. 5. When the main control unit 38 determines that the endoscope system 1 is not to be powered off (No), step S15 in FIG. 5 is executed again. When the main control unit 38 determines that the endoscope system 1 is to be powered off (Yes), the series of operations are ended.

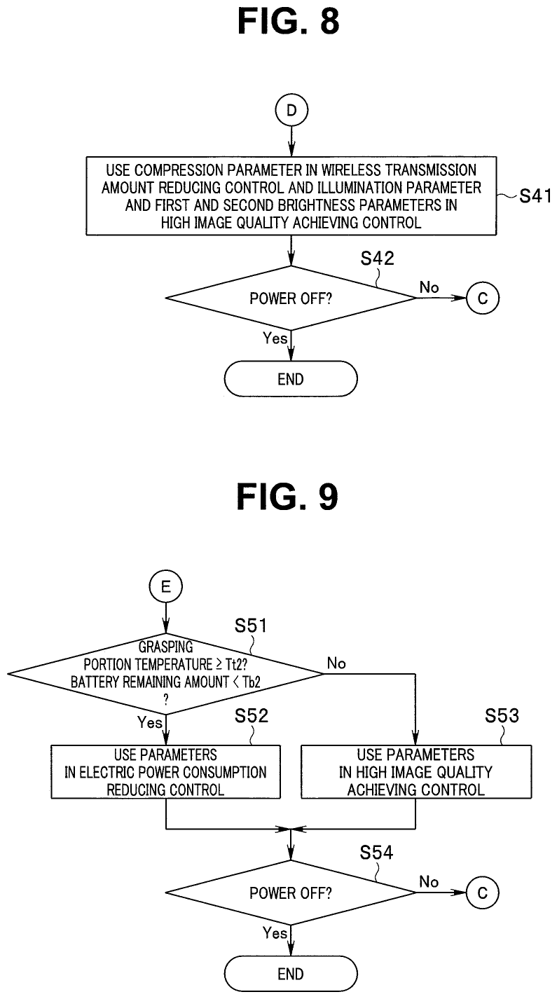

The series of steps illustrated in FIG. 8 correspond to operation of the endoscope system 1 when the electric power consumption reducing mode is not selected (No at step S31) at step S16, in other words, when the wireless transmission amount reducing mode and the high image quality achieving mode are selected at step S16. In this case, the compression processing unit 23A uses the compression parameter in the wireless transmission amount reducing control, namely, Cw, the illumination unit 22 uses the illumination parameter in the high image quality achieving control, namely, Bh, the filter processing unit 34A uses the first brightness parameter in the high image quality achieving control, namely, Fh, and the multiplication processing unit 34B uses the second brightness parameter in the high image quality achieving control, namely, Mh (step S41).

Subsequently, for example, the main control unit 38 determines whether to power off the endoscope system 1 (step S42). Contents of step S42 are the same as the contents of step S19 in FIG. 5. When the main control unit 38 determines that the endoscope system 1 is not to be powered off (No), step S15 in FIG. 5 is executed again. When the main control unit 38 determines that the endoscope system 1 is to be powered off (Yes), the series of operations are ended.

The series of steps illustrated in FIG. 9 correspond to operation of the endoscope system 1 when the electric power consumption reducing mode is selected (Yes at step S31) but the wireless transmission amount reducing mode is not selected (No at step S32) at step S16, in other words, when the electric power consumption reducing mode and the high image quality achieving mode are selected at step S16. In this case, when at least one of the requirement that the temperature of the grasping portion 2Ba, which is acquired at step S15 in FIG. 5 is equal to or higher than the second temperature threshold value Tt2 or the requirement that the remaining amount of the battery 25A, which is acquired at step S15 in FIG. 5 is smaller than the second battery threshold value Tb2 is satisfied (Yes at step S51), components of the endoscope 2 and the video processor 3 use the plurality of parameters in the electric power consumption reducing control, namely, Bp, Cp, Fp, and Mp (step S52).

When the requirement the temperature of the grasping portion 2Ba is equal to or higher than the second temperature threshold value Tt2 and the requirement that the remaining amount of the battery 25A is smaller than the second battery threshold value Tb2 are both not satisfied (No at step S51), components of the endoscope 2 and the video processor 3 use the plurality of parameters in the high image quality achieving control, namely, Bh, Ch, Fh, and Mh (step S53).

After step S52 or S53 is executed, for example, the main control unit 38 determines whether to power off the endoscope system 1 (step S54). Contents of step S54 are the same as the contents of step S19 in FIG. 5. When the main control unit 38 determines that the endoscope system 1 is not to be powered off (No), step 515 in FIG. 5 is executed again. When the main control unit 38 determines that the endoscope system 1 is to be powered off (Yes), the series of operations are ended.

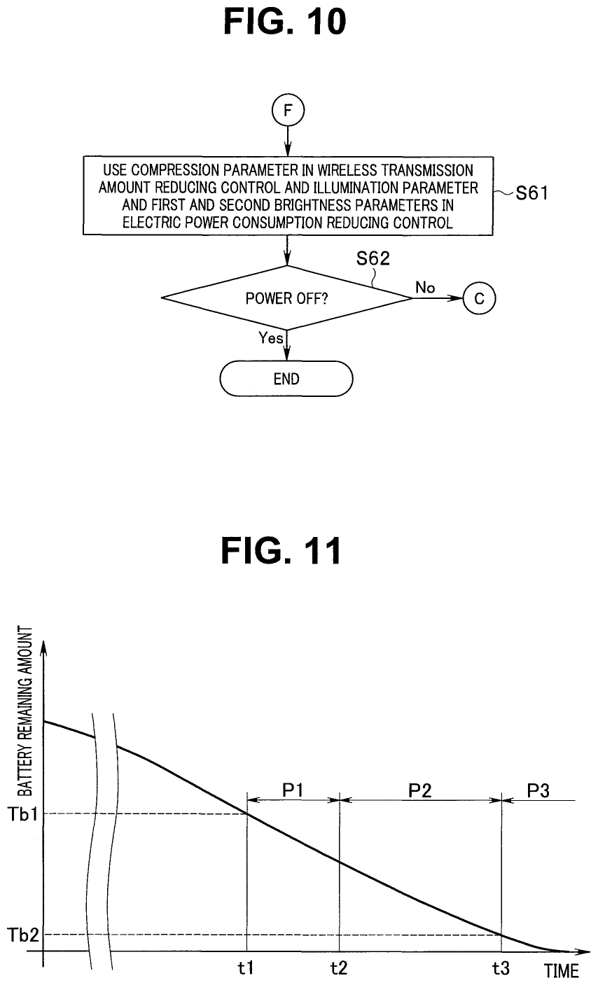

The series of steps illustrated in FIG. 10 correspond to operation of the endoscope system 1 when the electric power consumption reducing mode is selected (Yes at step S31), the wireless transmission amount reducing mode is selected (Yes at step S32), but the high image quality achieving mode is not selected (No at step S33) at step S16. In this case, the compression processing unit 23A uses the compression parameter in the wireless transmission amount reducing control, namely, Cw, the illumination unit 22 uses the illumination parameter in the electric power consumption reducing control, namely, Bp, the filter processing unit 34A uses the first brightness parameter in the electric power consumption reducing control, namely, Fp, and the multiplication processing unit 34B uses the second brightness parameter in the electric power consumption reducing control, namely, Mp (step S61).

Subsequently, for example, the main control unit 38 determines whether to power off the endoscope system 1 (step S62). Contents of step S62 are the same as the contents of step S19 in FIG. 5. When the main control unit 38 determines that the endoscope system 1 is not to be powered off (No), step S15 in FIG. 5 is executed again. When the main control unit 38 determines that the endoscope system 1 is to be powered off (Yes), the series of operations are ended.

(Setting Example of Parameters)

Subsequently, a setting example of the parameters will be described below. In this example, the illumination parameter, the compression parameter, the first brightness parameter, and the second brightness parameter are each expressed by using a value of one to five inclusive. It is set that the illumination light amount is strongest when the value of the illumination parameter is one, and the illumination light amount is weakest when the value is five. In other words, it is set that an effect of the electric power consumption reducing control is weakest when the value of the illumination parameter is one, and the effect of the electric power consumption reducing control is strongest when the value is five.

It is set that the compression ratio is lowest when the value of the compression parameter is one, and the compression ratio is highest when the value is five. In other words, it is set that the effect of the electric power consumption reducing control or an effect of the wireless transmission amount reducing control is weakest when the value of the compression parameter is one, and the effect of the electric power consumption reducing control or the effect of the wireless transmission amount reducing control is strongest when the value is five.

It is set that the effect of the filter processing is weakest when the value of the first brightness parameter is one, and the effect of the filter processing is strongest when the value is five. It is set that the effect of the multiplication processing is weakest when the value of the second brightness parameter is one, and the effect of the multiplication processing is strongest when the value is five. Brightness of a correction target pixel is lowest when the effect of the filter processing or the multiplication processing is weakest, and is highest when the effect of the filter processing or the multiplication processing is strongest.

Hereinafter, default values are defined to be the values of the parameters when the endoscope scene is the detailed-check scene in the standard control. The default values are three. First, a setting example of the parameters in the standard control will be described with reference to Table 1. Table 1 presents the setting example of the parameters in the standard control when the endoscope scene is the detailed-check scene, the screening scene, and the external scene.

TABLE 1

| | Detailed- | | |
|---|---|---|---|
| | check | | |
| Parameter | scene | Screening scene | External scene |
| Illumination | 3 | 4 | 5 |
| parameter | | | |
| Compression | 3 | 4 | 5 |
| parameter | | | |
| First brightness | 3 | 4 | 5 |
| parameter | | | |
| Second brightness | 3 | 4 | 5 |
| parameter | | | |

The illumination parameter, the compression parameter, the first brightness parameter, and the second brightness parameter are set so that the image quality and the resolution of the endoscope image are at predetermined levels when the endoscope scene is the detailed-check scene in the standard control. Hereinafter, a case in which the endoscope scene is the detailed-check scene in the standard control is referred to as a reference state. In the external scene, the image quality and the resolution of the endoscope image may be low. Thus, in the external scene, the illumination parameter and the compression parameter are set so that consumption of electric power of the battery 25A is smallest, and the first and second brightness parameters are set in accordance with the setting of the illumination parameter and the compression parameter. in the screening scene, the image quality and the resolution of the endoscope image are higher than in the external scene, but the illumination parameter, the compression parameer, the first brightness parameter, and the second brightness parameter are set so that consumption of electric power of the battery 25A is smaller than in the detailed-check scene.

Subsequently, a setting example of the parameters in the electric power consumption reducing control, the wireless transmission amount reducing control, and the high image quality achieving control will be described with reference to Table 2. Table 2 presents the setting example of the parameters in the electric power consumption reducing control, the wireless transmission amount reducing control, and the high image quality achieving control.

TABLE 2

| | Electric | Wireless | High |
|---|---|---|---|
| | power | transmission | image |
| | consumption | amount | quality |
| | reducing | reducing | achieving |
| Parameter | control | control | control |
| Illumination | 3.5 | 3 | 2 |
| parameter | | | |
| Compression | 3.25 | 3.5 | 2 |
| parameter | | | |
| First brightness | 3.5 | 2.5 | 2 |
| parameter | | | |
| Second brightness | 3.5 | 3.5 | 2 |
| parameter | | | |

The illumination parameter in the electric power consumption reducing control, namely, Bp is set to a value (in Table 2, 3.5) with which the illumination light amount of the illumination unit 22 is smaller than in the reference state. The compression parameter in the electric power consumption reducing control, namely, Cp is set to a value (in Table 2, 3.25) with which the data amount of the compressed data is slightly smaller than in the reference state. The first brightness parameter in the electric power consumption reducing control, namely, Fp is set to a value (in Table 2, 3.5) with which the effect of the filter processing is stronger than in the reference state. The second brightness parameter in the electric power consumption reducing control, namely, Mp is set to a value (in Table 2, 3.5) with which the effect of the multiplication processing is stronger than in the reference state.

The illumination parameter in the wireless transmission amount reducing control is set to a value (in Table 2, 3) the same as in the reference state. The compression parameter in the wireless transmission amount reducing control, namely, Cw is set to a value (in Table 2, 3.5) with which the data amount of the compressed data is significantly smaller than in the reference state. The first brightness parameter in the wireless transmission amount reducing control, namely, Fw is set to a value (in Table 2, 2.5) with which the effect of the filter processing is weaker than in the reference state. The second brightness parameter in the wireless transmission amount reducing control, namely, Mw is set to a value (in Table 2, 3.5) with which the effect of the multiplication processing is stronger than in the reference state.

Note that comparison for the same effect of the electric power consumption reducing control indicates that the illumination light amount change processing can typically reduce decrease of the resolution of the endoscope image as compared to the compression amount change processing. As indicated in Table 2, it is possible to reduce decrease of the resolution of the endoscope image in the electric power consumption reducing control by setting the compression parameter in the electric power consumption reducing control to a value with which the data amount of the compressed data is slightly smaller.

Typically, the resolution of the endoscope image decreases as the compression ratio increases, in other words, as the data amount of the compressed data decreases. In addition, the resolution of the endoscope image decreases as the effect of the filter processing increases. However, as indicated in Table 2, it is possible to reduce decrease of the resolution of the endoscope image in the wireless transmission amount reducing processing by setting the first brightness parameter in the wireless transmission amount reducing processing to a value with which the effect of the filter processing is weaker. In addition, it is possible to reduce decrease of the effect of the brightness correction processing in the wireless transmission amount reducing processing by setting the second brightness parameter in the wireless transmission amount reducing processing to a value with which the effect of the multiplication processing is stronger.

The illumination parameter in the high image quality achieving control, namely, Bh is set to a value (in Table 2, 2) with which the illumination light amount of the illumination unit 22 is larger than in the reference state. The compression parameter in the high image quality achieving control, namely, Ch is set to a value (in Table 2, 2) with which the data amount of the compressed data is larger than in the reference state. The first brightness parameter in the high image quality achieving control, namely, Fh is set to a value (in Table 2, 2) with which the effect of the filter processing is weaker than in the reference state. The second brightness parameter in the high image quality achieving control, namely, Mh is set to a value (in Table 2, 2) with which the effect of the multiplication processing is weaker than in the reference state.

(Operations and Effects)

Subsequently, operations and effects of the endoscope system 1 and the parameter control device 5 according to the present embodiment will be described below. In the present embodiment, the operation mode selection unit 52 of the parameter control device 5 determines a plurality of pieces of information collected by the data collection unit 51, thereby selecting one or more of the operation modes among the electric power consumption reducing mode, the wireless transmission amount reducing mode, the high image quality achieving mode, and the standard. mode. The parameter determination unit 53 of the parameter control device 5 determines a plurality of parameters based on the one or more operation modes selected by the operation mode selection unit 52. As described above, in accordance with contents of operation mode selection, the plurality of parameters are selected from among parameters defined in advance. In the present embodiment, the parameter determination unit 53 determines the plurality of parameters with control priority taken into account. The control priority is defined to prevent occurrences of battery exhaustion and wireless communication blackout. With this configuration, according to the present embodiment, it is possible to select one or more operation modes, while preventing occurrences of battery exhaustion and wireless communication blackout.

The control priority will be described below. The description will be first made on, with reference to FIG. 11, a case in which the operation mode selection unit 52 selects the electric power consumption reducing mode and the wireless transmission amount reducing mode, and a case in which the operation mode selection unit 52 selects the electric power consumption reducing mode, the wireless transmission amount reducing mode, and the high image quality achieving mode. FIG. 11 is an explanatory diagram schematically illustrating change of the remaining amount of the battery 25A. In FIG. 11, a horizontal axis represents time, and a vertical axis represents the remaining amount of the battery 25A. In FIG. 11, symbol Tb1 represents the first battery threshold value, and symbol Tb2 represents the second battery threshold value.

In FIG. 11, symbols t1, t2, and t3 represent time points. Time point t1 is a time point at which the remaining amount of the battery 25A becomes equal to the first battery threshold value Tb1. Time point t2 is a time point at which the video recording information acquisition unit 51A acquires the information for starting the video recording of the endoscope image. Time point t3 is a time point at which the remaining amount of the battery 25A becomes equal to the second battery threshold value Tb2. A duration earlier than the time point t1 corresponds to a state in which the remaining amount of the battery 25A is sufficient. A duration after the time point t3 corresponds to a state in which the remaining amount of the battery 25A is about to run out.

Assume that the operation mode selection unit 52 selects the wireless transmission amount reducing mode in the duration earlier than the time point t1. When the time point t1 is passed and the remaining amount of the battery 25A becomes smaller than the first battery threshold value Tb1, the operation mode selection unit 52 selects the electric power consumption reducing mode and the wireless transmission amount reducing. mode. In this case, as described above, the parameter determination unit 53 determines, as the plurality of parameters, the illumination parameter and the first and second brightness parameters in the high image quality achieving control and the compression parameter in the wireless transmission amount reducing control. Accordingly, the illumination light amount change processing, the filter processing, and the multiplication processing in the electric power consumption reducing control, and the compression amount change processing in the wireless transmission amount reducing control are executed in effect in duration P1 from the time point t1 to the time point t2. In other words, in the duration P1, the electric power consumption reducing control is prioritized for the illumination light amount change processing, the filter processing, and the multiplication processing, and the wireless transmission amount reducing control is prioritized for the compression amount change processing.

At the time point t2, the operation mode selection unit 52 selects the electric power consumption reducing mode, the wireless transmission amount reducing mode, and the high image quality achieving mode. At the time point t2, the remaining amount of the battery 25A is smaller than the first battery threshold value Th1 and equal to or larger than the second battery threshold value Tb2. In this case, the parameter determination unit 53 determines, as the plurality of parameters, the illumination parameter and the first and second brightness parameters in the high image quality achieving control and the compression parameter in the wireless transmission amount reducing control as described above. Accordingly, the illumination light amount change processing, the filter processing, and the multiplication processing in the high image quality achieving control, and the compression amount change processing in the wireless transmission amount reducing control are executed in effect in duration P2 from the time point t2 to the time point t3. In other words, in the duration P2, the high image quality achieving control is prioritized for the illumination light amount change processing, the filter processing, and the multiplication processing, and the wireless transmission amount reducing control is prioritized for the compression amount change processing.

When the time point t3 is passed and the remaining amount of the battery 25A becomes smaller than the second battery threshold value Tb2 while a condition that the operation mode selection unit 52 selects the electric power consumption reducing mode, the wireless transmission amount reducing mode, and the high image quality achieving mode is satisfied, the parameter determination unit 53 determines, as the plurality of parameters, the illumination parameter and the first and second brightness parameters in the high image quality achieving control and the compression parameter in the wireless transmission amount reducing control as described above. Accordingly, the illumination light amount change processing, the filter processing, and the multiplication processing in the electric power consumption reducing control, and the compression amount change processing in the wireless transmission amount reducing control are executed in effect in duration P3 after the time point t3. In other words, in the duration P3, the electric power consumption reducing control is prioritized for the illumination light amount change processing, the filter processing, and the multiplication processing, and the wireless transmission amount reducing control is prioritized for the compression amount change processing.

In the duration P1, the electric power consumption reducing control and the wireless transmission amount reducing control are both prioritized. Accordingly, it is possible to prevent battery exhaustion and wireless communication blackout. In the duration P2, the high image quality achieving control is prioritized over the electric power consumption reducing control, and the wireless transmission amount reducing control is prioritized. Accordingly, it is possible to achieve high image quality of the endoscope image and prevent wireless communication blackout in a situation in which the electric power consumption reducing control is executed but the remaining amount of the battery 25A is not about to run out. In the duration P3, the electric power consumption reducing control is prioritized over the high image quality achieving control, and the wireless transmission amount reducing control is prioritized. Accordingly, it is possible to prevent battery exhaustion and wireless communication blackout in a situation in which the remaining amount of the battery 25A is about to run out.

The priority when the remaining amount of the battery 25A is changed is described above. The above description is also applicable to a case in which the temperature of the grasping portion 2Ba is changed. In this case, it is possible to prevent high temperature of the grasping portion 2Ba instead of preventing battery exhaustion.

Subsequently, priority when the operation mode selection unit 52 selects the electric power consumption reducing mode and the high image quality achieving mode will be described below. The priority in this case is the same as the priority described with reference to FIG. 11 except priority for the compression amount change processing. Note that, as for the compression amount change processing, the high image quality achieving control is prioritized in a situation in which the high image quality achieving control is prioritized over the electric power consumption reducing control for the processing other than the compression amount change processing, and the electric power consumption reducing control is prioritized in a situation in which the electric power consumption reducing control is prioritized over the high image quality achieving control for the processing other than the compression amount change processing.

Subsequently, priority when the operation mode selection unit 52 selects the wireless transmission amount reducing mode and the high image quality achieving mode will be described below. In this case, the high image quality achieving control is prioritized for the illumination light amount change processing, the filter processing, and the multiplication processing, and the wireless transmission amount reducing control is prioritized for the compression amount change processing. Accordingly, it is possible to prevent wireless communication blackout and achieve high image quality of the endoscope image.

### Second Embodiment

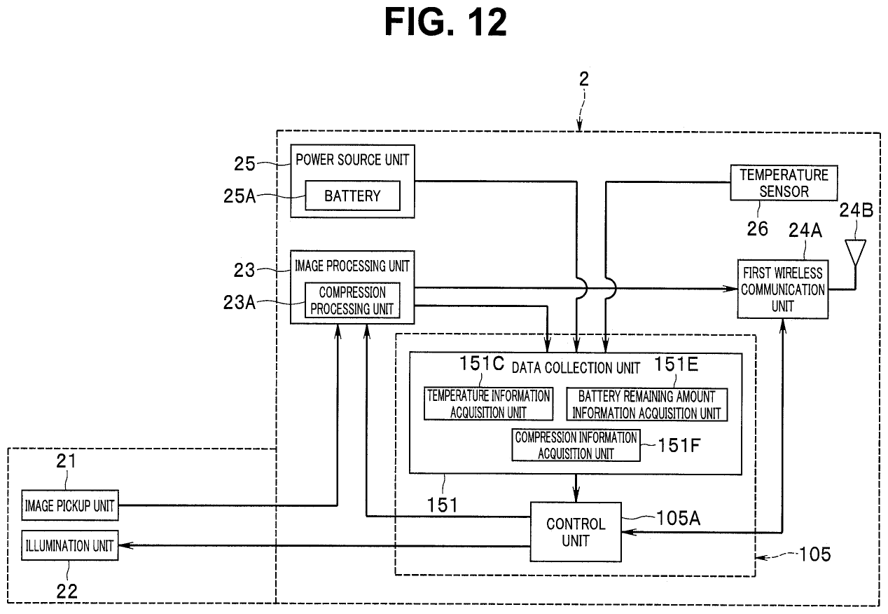

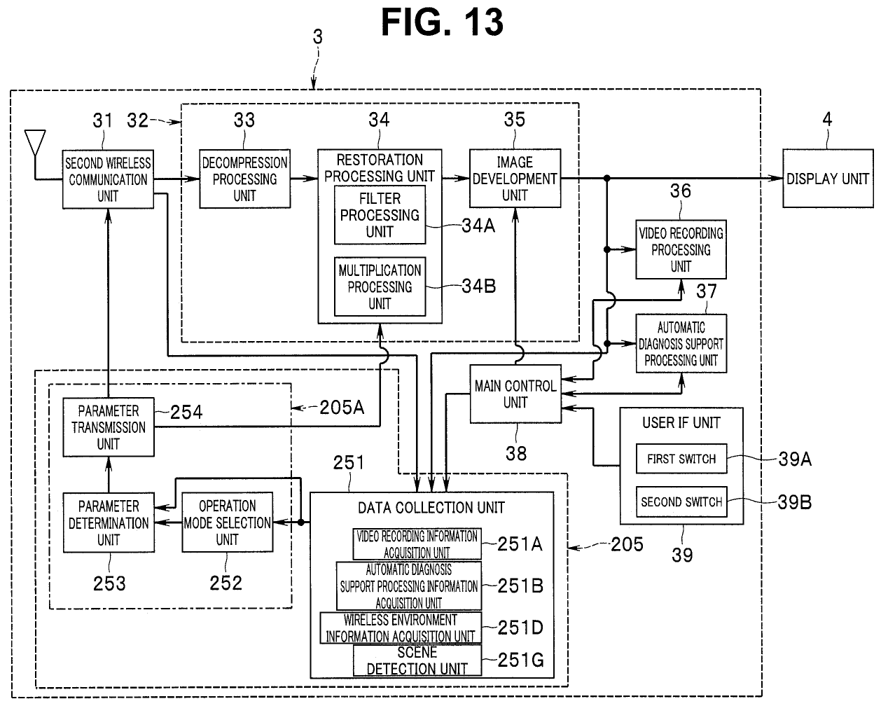

Subsequently, an endoscope system according to a second embodiment of the present invention will be described below with reference to FIGS. 12 and 13. FIG. 12 is a functional block diagram illustrating a configuration of all endoscope and a first part of a parameter control device in the endoscope system according to the present embodiment. FIG. 13 is a functional block diagram illustrating a configuration of a video processor and a second part of the parameter control device in the endoscope system according to the present embodiment. As illustrated in FIGS. 12 and 13, the endoscope system according to the present embodiment includes the parameter control device according to the present embodiment in place of the parameter control device 5 according to the first embodiment. The parameter control device according to the present embodiment includes a first part 105 provided in the endoscope 2, and a second part 205 provided in the video processor 3.

As illustrated in FIG. 12, the first part 105 of the parameter control device includes a data collection unit 151 and a control unit 105A. The data collection unit 151 includes a temperature information acquisition unit 151C, a battery remaining amount information acquisition unit 151E, and a compression information acquisition unit 151E in other words, the temperature information acquisition unit 151C and the battery remaining amount information acquisition unit 151E are provided in the endoscope 2. Functions of the temperature information acquisition unit 151C, the battery remaining amount information acquisition unit 151E, and the compression information acquisition unit 151F are the same as functions of the temperature information acquisition unit 51C, the battery remaining amount information acquisition unit 51E, and the compression information acquisition unit 51F, respectively, in the first embodiment.

The data collection unit 151 outputs, to the control unit 105A, information related to the temperature of the grasping portion 2Ba, which is acquired by the temperature information acquisition unit 151C, information related to the remaining amount of the battery 25A, which is acquired by the battery remaining amount information acquisition unit 151E, and information related to the compression processing, which is acquired by the compression information acquisition unit 151F. The control unit 105A outputs the plurality of pieces of information acquired by the data collection unit 151 to the second part 205 of the parameter control device through wireless communication between the endoscope 2 and the video processor 3.

As illustrated in FIG. 13, the second part 205 of the parameter control device includes a data collection unit 251, an operation mode selection unit 252, a parameter determination unit 253, and a parameter transmission unit 254. The operation mode selection unit 252, the parameter determination unit 253, and the parameter transmission unit 254 are included in a control unit 205A as a main part of the parameter control device. In other words, the operation mode selection unit 252 and the parameter determination unit 253 are provided in the video processor 3.

The data collection unit 251 includes a video recording information acquisition unit 251A, an automatic diagnosis support processing information acquisition unit 251B, a wireless environment information acquisition unit 251D, and a scene detection unit 251G. In other words, the video recording information acquisition unit 251A, the automatic diagnosis support processing information acquisition unit 251B, and the wireless environment information acquisition unit 251D are provided in the video processor 3.

Functions of the video recording information acquisition unit 251A and the automatic diagnosis support processing information acquisition unit 251B are basically the same as functions of the video recording information acquisition unit 51A and the automatic diagnosis support processing information acquisition unit 51B, respectively, in the first embodiment. Note that, in the present embodiment, the main control unit 38 of the video processor 3 outputs, to the data collection unit 251, information for starting or stopping the video recording of the endoscope image and information for starting or stopping the automatic diagnosis support processing. Accordingly, the video recording information acquisition unit 251A can acquire the information for starting or stopping the video recording of the endoscope image, and the automatic diagnosis support processing information acquisition unit 251B can acquire the information for starting or stopping the automatic diagnosis support processing.

Functions of the wireless environment information acquisition unit 251D are basically the same as functions of the wireless environment information acquisition unit 51D in the first embodiment. Note that, in the present embodiment, the second wireless communication unit 31A includes a non-illustrated environment detection circuit configured to detect the state of the wireless environment. The wireless environment information acquisition unit 251D acquires, as the information related to the wireless environment, a result of the detection by the environment detection circuit of the second wireless communication unit 31A or a forwardable data amount calculated based on the result of the detection by the environment detection circuit. Note that, in the present embodiment, the first wireless communication unit 24A may or may not include an environment detection circuit. In the former case, the first wireless communication unit 24A outputs information related to the wireless environment, which is detected by the environment detection circuit to the second part 205 of the parameter control device through wireless communication between the endoscope 2 and the video processor 3.

Functions of the scene detection unit 251G are basically the same as functions of the scene detection unit 51G in the first embodiment. Note that, in the present embodiment, the image processing unit 32 outputs, as information related to an endoscope scene, image data for detecting an endoscope scene to the second part 205 of the parameter control device. in an example illustrated in FIG. 13, the scene detection unit 251G receives the endoscope image outputted from the image development unit 35 of the image processing unit 32. The scene detection unit 251G detects an endoscope scene by analyzing acquired image data, in other words, the endoscope image.

The data collection unit 251 receives a plurality of pieces of data collected by the data collection unit 151 and outputted from the control unit 105A. Accordingly, the data collection unit 251 acquires the plurality of pieces of information acquired by the data collection unit 151 in effect.

The operation mode selection unit 252 determines the plurality of pieces of information acquired by the data collection unit 251 (including the plurality of pieces of information acquired by the data collection unit 151), thereby selecting one or more operation modes. A method of the operation mode selection is the same as in the first embodiment.

The parameter determination unit 253 determines a plurality of parameters based on the one or more operation modes selected by the operation mode selection unit 252. A method of the parameter determination is the same as in the first embodiment.

The parameter transmission unit 254 transmits the plurality of parameters determined by the parameter determination unit 253 to components of the endoscope 2 and the video processor 3. Specifically, the parameter transmission unit 254 transmits an illumination parameter and a compression parameter to the control unit 105A, transmits a first brightness parameter to the filter processing unit 34A of the restoration processing unit 34, and transmits a second brightness parameter to the multiplication processing unit 34B of the restoration processing unit 34. The control unit 105A outputs the received illumination parameter to the illumination unit 22, and outputs the received compression parameter to the compression processing unit 23A.

In the present embodiment, the control unit 205A as the main part of the parameter control device is provided in the video processor 3. With this configuration, according to the present embodiment, consumption of electric power of the battery 25A can be reduced as compared to a configuration in which the main part of the parameter control device is provided in the endoscope 2.

Other configurations, operations, and effects in the present embodiment are the same as the configurations, the operations, and the effects in the first embodiment.

The present invention is not limited to the above-described embodiments but may be provided with various kinds of changes, modifications, and the like without changing the gist of the present invention. For example, each parameter control device of the present invention may be a device separated from the endoscope 2 and the video processor 3.

The wireless environment information acquisition unit and the scene detection unit of each data collection unit may be provided in both the endoscope 2 and the video processor 3.

In addition to the illumination light amount change processing, the compression amount change processing, and the brightness correction processing, the electric power consumption reducing control may include warning processing that warns a user that the electric power consumption reducing control is to be executed. Similarly, in addition to the compression amount change processing and the brightness correction processing, the wireless transmission amount reducing control may include warning processing that warns the user that the wireless transmission amount reducing control is to be executed. The warning processing may be, for example, processing that causes the display unit 4 to display characters or the like indicating that the electric power consumption reducing control or the wireless transmission amount reducing control is in execution.

What is claimed is:

1. A video processor comprising a processor, wherein
the processor is configured to:
acquire at least two pieces of information of information related to temperature of a grasping portion of an endoscope, information related to wireless environment of wireless communication that transmits and receives image data obtained through image pickup by the endoscope, information related to a remaining amount of a battery of the endoscope, or information for starting video recording of an endoscope image; and
control a plurality of parameters, and
the processor
selects, based on the at least two pieces of information, one or more operation modes of a plurality of operation modes that define operation contents of the endoscope and the video processor, and
determines the plurality of parameters based on the one or more selected operation modes.

2. The video processor according to claim 1, wherein the plurality of operation modes include
an electric power consumption reducing mode in which electric power consumption reducing control is performed to reduce electric power supplied from the battery,
a wireless transmission amount reducing mode in which wireless transmission amount reducing control is performed to reduce a data amount of the wireless communication,
a high image quality achieving mode in which high image quality achieving control is performed to achieve high image quality of the endoscope image, and
a standard mode in which neither the electric power consumption reducing control, the wireless transmission amount reducing control, nor the high image quality achieving control is performed but standard control is performed to control the endoscope and the video processor.

3. The video processor according to claim 2, wherein the processor is able to further acquire information for starting automatic diagnosis support processing using the endoscope image.

4. The video processor according to claim 3, wherein
the processor selects the electric power consumption reducing mode when at least one of a condition that the temperature of the grasping portion is equal to or higher than a predetermined first temperature threshold value or a condition that the remaining amount of the battery is smaller than a predetermined first battery threshold value is satisfied.
the processor selects the wireless transmission amount reducing mode when a forwardable data amount of the wireless communication is smaller than a predetermined threshold value,
the processor selects the high image quality achieving mode when the processor acquires at least one of the information for starting video recording of an endoscope image or the information for starting automatic diagnosis support processing, and
the processor selects the standard mode when none of selection conditions of the electric power consumption reducing mode, the wireless transmission amount reducing mode, and the high image quality achieving mode are satisfied.

5. The video processor according to claim 3, wherein
the processor is further configured to:
generate compressed data by compressing the image data,
generate decompressed image data corresponding to the image data by decompressing the compressed data, and
perform predetermined image processing on the decompressed image data,
the predetermined image processing includes brightness correction processing that corrects brightness of the decompressed image data,
the electric power consumption reducing control and the high image quality achieving control each include illumination light amount change processing that changes an illumination light amount of an illumination element, compression amount change processing that changes a data amount of the compressed data, and the brightness correction processing,
the wireless transmission amount reducing control includes the compression amount change processing and the brightness correction processing,
the illumination light amount change processing is processing in which an illumination parameter that defines the illumination light amount of the illumination element is used,
the compression amount change processing is processing in which a compression parameter that defines the data amount of the compressed data is used, and
the brightness correction processing is processing in which a brightness parameter that defines a relation between brightness of the decompressed image data. before correction and brightness of the decompressed image data after correction is used.

6. The video processor according to claim 5, wherein
the illumination parameter in the electric power consumption reducing control is defined so that the illumination light amount is smaller than in the standard control,
the illumination parameter in the high image quality achieving control is defined so that the illumination light amount is larger than in the standard control,
the compression parameter in the electric power consumption reducing control and the compression parameter in the wireless transmission amount reducing control are defined so that the data amount of the compressed data is smaller than in the standard control,
the compression parameter in the high image quality achieving control is defined so that the data amount of the compressed data is larger than in the standard control,
the brightness parameter in the electric power consumption reducing control is defined so that an effect of the brightness correction processing that brightens the endoscope image is stronger than in the standard control,
the brightness parameter in the wireless transmission amount reducing control is defined so that brightness of the endoscope image is corrected with decrease of a resolution of the endoscope image being reduced as compared to the standard control, and
the brightness parameter in the high image quality achieving control is defined so that the effect of the brightness correction processing is weaker than in the standard control.

7. The video processor according to claim 5, wherein
the processor determines, as the plurality of parameters, the illumination parameter, the compression parameter, and the brightness parameter in the high image quality achieving control when the processor selects the electric power consumption reducing mode and the high image quality achieving mode and the temperature of the grasping portion is lower than a predetermined second temperature threshold value, and
the processor determines, as the plurality of parameters, the illumination parameter, the compression parameter, and the brightness parameter in the electric power consumption reducing control when the processor selects the electric power consumption reducing mode and the high image quality achieving mode and the temperature of the grasping portion is equal to or higher than the second temperature threshold value.

8. The video processor according to claim 5, wherein
the processor determines, as the plurality of parameters, the illumination parameter, the compression parameter, and the brightness parameter in the high image quality achieving control when the processor selects the electric power consumption reducing mode and the high image quality achieving mode and the remaining amount of the battery is equal to or larger than a predetermined second battery threshold value, and
the processor determines, as the plurality of parameters, the illumination parameter, the compression parameter, and the brightness parameter in the electric power consumption reducing control when the processor selects the electric power consumption reducing mode and the high image quality achieving mode and the remaining amount of the battery is smaller than the second battery threshold value.

9. The video processor according to claim 5, wherein
the processor determines, as the plurality of parameters, the illumination parameter and the brightness parameter in the high image quality achieving control and the compression parameter in the wireless transmission amount reducing control when the processor selects the wireless transmission amount reducing mode and the high image quality achieving mode.

10. The video processor according to claim 5, wherein
the processor determines, as the plurality of parameters, the illumination parameter and the brightness parameter in the high image quality achieving control and the compression parameter in the wireless transmission amount reducing control when the processor selects the electric power consumption reducing mode, the wireless transmission amount reducing mode, and the high image quality achieving mode and the temperature of the grasping portion is lower than a predetermined second temperature threshold value, and
the processor determines, as the plurality of parameters, the illumination parameter and the brightness parameter in the electric power consumption reducing control and the compression parameter in the wireless transmission amount reducing control when the processor selects the electric power consumption reducing mode, the wireless transmission amount reducing mode, and the high image quality achieving mode and the temperature of the grasping portion is equal to or higher than the second temperature threshold value.

11. The video processor according to claim 5, wherein
the processor determines, as the plurality of parameters, the illumination parameter and the brightness parameter in the high image quality achieving control and the compression parameter in the wireless transmission amount reducing control when the processor selects the electric power consumption reducing mode, the wireless transmission amount reducing mode, and the high image quality achieving mode and the remaining amount of the battery is equal to or larger than a predetermined second battery threshold value, and
the processor determines, as the plurality of parameters, the illumination parameter and the brightness parameter in the electric power consumption reducing control and the compression parameter in the wireless transmission amount reducing control when the processor selects the electric power consumption reducing mode, the wireless transmission amount reducing mode, and the high image quality achieving mode and the remaining amount of the battery is smaller than the second battery threshold value.

12. An endoscope system comprising an endoscope, a video processor, and a processor, wherein
the processor is configured to:
acquire information related to temperature of a grasping portion of the endoscope;
acquire information related to wireless environment of wireless communication that transmits and receives image data obtained through image pickup by the endoscope;
acquire information related to a remaining amount of a battery of the endoscope;
acquire information for starting video recording of an endoscope image; and
acquire information for starting automatic diagnosis support processing,
the acquisition of the information related to the wireless environment, the acquisition of the information for starting video recording of an endoscope image, and the acquisition of the information for starting automatic diagnosis support processing are executed by at least one of the endoscope or the video processor, and
the acquisition of the information related to the temperature of the grasping portion and the acquisition of the information related to the remaining amount of the battery are executed by the endoscope.

13. The endoscope system according to claim 12, wherein
selection of, based on at least two pieces of information of the plurality of pieces of information, one or more operation modes of a plurality of operation modes that define operation contents of the endoscope and the video processor and determination of a plurality of parameters based on the one or more selected operation modes are executed by the endoscope.

14. An endoscope comprising a processor, wherein
the processor is configured to:
acquire at least two pieces of information of information related to temperature of a grasping portion of the endoscope, information related to wireless environment of wireless communication that transmits and receives image data obtained through image pickup by the endoscope, information related to a remaining amount of a battery of the endoscope, or information for starting video recording of an endoscope image, and
control a plurality of parameters, and
the processor
selects, based on the at least two pieces of information, one or more operation modes of a plurality of operation modes that define operation contents of the endoscope and a video processor, and
determines the plurality of parameters based on the one or more selected operation modes.

15. An image processing method of processing image data acquired by an image pickup device of an endoscope, the image processing method comprising
acquiring at least two pieces of information of information related to temperature of a grasping portion of the endoscope, information related to wireless environment of wireless communication that transmits and receives the image data, information related to a remaining amount of a battery of the endoscope, or information for starting video recording of an endoscope image;
selecting, based on the at least two pieces of information, one or more operation modes of a plurality of operation modes that define operation contents of the endoscope and a video processor; and
determining a plurality of parameters based on the one or more selected operation modes.
